(19) United States
(12) Patent Application Publication
(10) Pub. No.: US 2023/0372016 A1
(43) Pub. Date: Nov. 23, 2023

(54) APPARATUS AND METHODS FOR DETERMINING AN OPTIMIZED IMPLANT POSITION USING A KINEMATIC AND INVERSE DYNAMICS MODEL AND APPLYING MOTION CAPTURE DATA

(71) Applicant: INTELLIJOINT SURGICAL INC., KITCHENER (CA)

(72) Inventors: Behzad Danaei, London (CA); John Joseph McPhee, Waterloo (CA); Joseph Arthur Schipper, Kitchener (CA); Andre Novomir Hladio, Waterloo (CA)

(21) Appl. No.: 18/198,970

(22) Filed: May 18, 2023

Related U.S. Application Data
(60) Provisional application No. 63/343,955, filed on May 19, 2022.

(51) Int. Cl. A61B 34/10; A61F 2/34; A61B 5/11; A61B 90/00

(52) U.S. Cl. CPC A61B 34/10; A61F 2/34; A61B 5/1128; A61B 90/36; A61B 2034/105; A61B 2034/107; A61B 2034/108; A61B 2034/104; A61B 2034/2057

(57) ABSTRACT

Methods are disclosed for determining an optimized implant position using a kinematic and inverse dynamics model to model one or more outcome factors for a joint reconstruction of a patient. Motion capture data and geometric and inertial parameter data are applied to the model to optimize one or more outcome parameters associated with the one or more outcome factors to generate the optimized implant position. The optimized implant position is provided for use by a surgical planning system and/or an intra-operative surgical navigation system. A related apparatus is also disclosed comprising a storage device coupled to a processor that is configured to execute instructions stored on the storage device to perform the methods.

### CROSS-REFERENCE TO RELATED APPLICATION

This application claims the domestic benefit of U.S. Provisional Application No. 63/343,955 filed May 19, 2022, the entire contents of which are incorporated herein by reference.

### FIELD OF THE INVENTION

This application relates to apparatus and related methods for planning a surgical procedure, such as an anatomical reconstruction, to improve surgical outcome and more particularly to apparatus and methods for determining an optimized implant position using a kinematic and inverse dynamics model and applying motion capture data.

### BACKGROUND

In a Total Hip Arthroplasty (THA), the head and stem of the femur are removed and replaced with artificial components. In addition, the acetabulum is reamed and an artificial acetabular cup and liner are inserted. The artificial femoral head and stem and the acetabular cup and liner form an implant system. The desired anatomical reconstruction is defined by implants (size, shape, type) and their positions relative to the patient's anatomy. Cup positioning (orientation and placement), cup size, femoral version, femoral head size, femoral stem size, femoral stem offset, and neck-shaft angle are important elements of the implant system and can influence the patient's outcome following THA, including pain, mobility, range of motion and the risk of hip dislocation. There exists a need to improve positioning of the artificial joint components during THA.

The general goal of surgical planning systems is to improve patient outcomes by planning for a desirable anatomical reconstruction. Factors related to implant positioning known to affect the patient's outcome include edge loading, implant impingement, bony impingement, bone-on-implant impingement, and soft tissue impingement. Existing surgical planning systems provide functionality to plan for desired anatomical reconstruction based on medical images of a patient and kinematic modeling techniques. However, in the context of THA, these approaches account only for some factors known to affect patient outcomes, such as the various kinds of impingement, but usually do not account for hip contact forces and edge-loading. Some studies have attempted to include edge-loading to determine optimal cup orientation, but they require force plate measurements, which are not suitable for clinical settings due to the time and expense required. As a result, in clinical settings, the effect of full-body motion and body dynamics are neglected.

There exists a need to improve surgical planning systems. It may be desirable to incorporate full-body motion and body dynamics using kinematic and inverse dynamics modeling in surgical planning that do not require force plate measurements.

### SUMMARY

Methods are disclosed for determining an optimized implant position using a kinematic and inverse dynamics (KID) model to model one or more outcome factors for a joint reconstruction of a patient. Motion capture (MoCap) data and geometric and inertial parameter data are applied to the model to optimize one or more outcome parameters associated with the one or more outcome factors to generate the optimized implant position. The optimized implant position is provided for use by a surgical planning system and/or an intra-operative surgical navigation system. A related apparatus is also disclosed comprising a storage device coupled to a processor that is configured to execute instructions stored on the storage device to perform the methods.

There is provided a method comprising storing a KID model of a patient, the model comprising, for each of one or more outcome factors that are modeled, one or more respective outcome parameters of an implant system for a joint reconstruction of a joint of the patient; generating, in accordance with MoCap data for the patient's movement and geometric and inertial parameter data for the patient applied to the model, an optimized position of the implant system for the patient by optimizing the one or more respective outcome parameters of at least one of the outcome factors; and presenting the optimized position in association with a medical image comprising a bone of the patient associated with the joint.

In an embodiment, the one or more outcome factors comprises any of: an edge-loading factor, an implant impingement factor, a bony impingement factor, a bone-on-implant impingement factor and a soft tissue impingement factor.

In an embodiment, the one or more outcome factors comprises an edge loading factor and an implant impingement factor.

In an embodiment, generating the optimized position of the implant system comprises performing a mathematical optimization which minimizes at least some of the respective outcome parameters.

In an embodiment, the one or more outcome factors comprises a plurality (N) of outcome factors; and generating the optimized position comprises constraining the one or more respective parameters respectively associated with N-1 of one or more outcome factors while optimizing the one or more respective outcome parameters associated with one of N outcome factors that is unconstrained.

In an embodiment, generating the optimized position comprises performing, for at least two of the outcome factors, a combined optimization of one or more respective outcome parameters respectively associated with at least two of the outcome factors.

In an embodiment, the model performs an estimation of ground reaction forces and moments without the need for force plate measurements.

In an embodiment, the MoCap data is in a first coordinate system, and the medical image is in a second coordinate system; and the method comprises performing a registration of the first coordinate system and the second coordinate system for presenting the optimized position in association with the medical image.

In an embodiment, the MoCap data is generated by a MoCap system. In an embodiment, the MoCap system uses any of the following technologies: optical marker-based motion capture; marker-less motion capture based on video feed; inertial sensors; and inertial measurement units.

In an embodiment, the MoCap system comprises one or more optical and/or inertial devices having radiopaque features associated with the first coordinate system, and wherein the medical image includes an image of the radiopaque features of the one or more optical and/or inertial devices as coupled to the patient for generating the MoCap data. In an embodiment, the radiopaque features are one of: optical markers coupled to the patient; an inertial device with radiopaque features embedded within, wherein the radiopaque features comprise at least three retroreflective markers with a known position relative to the MoCap data coordinate system.

In an embodiment, performing the registration comprises calculating a transformation between the first coordinate system and the second coordinate system using locations of the radiopaque features measurable within the MoCap data in the first coordinate system and respective locations of the radiopaque features measurable within the medical image in the second coordinate system. In an embodiment, the respective locations of the radiopaque features in the second coordinate system are measured using image processing of the medical image.

In an embodiment, the MoCap data may include anatomical landmark data for the purpose of performing a registration of the first coordinate system and the second coordinate system; the medical image includes corresponding anatomical landmark data; and performing the registration comprises calculating a transformation between the first coordinate system and the second coordinate system using locations of the anatomical landmark data in the first coordinate system and respective locations of the corresponding anatomical landmark data in the second coordinate system.

In an embodiment, the geometric and inertial parameter data may comprise one or more of: body segment lengths, body segment masses, body segment centers of mass, and an inertia matrix.

In an embodiment, the joint may be a hip and the implant system may be an artificial hip joint comprising any of: a cup; a liner; a stem; and a femoral head.

In an embodiment, the optimized position of the implant system is associated with any of: a cup orientation; a cup translational position; a femoral version; a femoral head size; a cup size; a stem size; a stem offset; and a femoral neck-shaft angle.

In an embodiment, the optimized position of the implant system is associated with any of: a cup orientation; a cup translational position; and a stem offset.

In an embodiment, the patient image comprises one of an x-ray, a magnetic resonance imaging (MRI) scan, a computed tomography (CT) scan, and an ultrasound scan.

In an embodiment, the method comprises providing the optimized implant position for use by either or both of a surgical planning system, and an intra-operative navigation system.

There is provided a method comprising: storing a KID model of a patient, the model comprising, for each of one or more outcome factors that are modeled, one or more respective outcome parameters of an implant system for a joint reconstruction of the patient; generating an optimized position of the implant system for the patient in accordance with the model and using MoCap data for the patient's movement and geometric and inertial parameter data for the patient wherein: the optimized position is generated by optimizing the one or more respective outcome parameters of at least one of the outcome factors, the MoCap data is in a first coordinate system, and the MoCap data comprises MoCap landmark data associated with anatomical landmarks of the patient spanning the first coordinate system; and providing the optimized position, in the first coordinate system, to an intra-operative navigation system, the system configured for use when registered together with the first coordinate system for executing the optimal implant position.

There is provided a method comprising: storing a KID model of a patient, the model modeling a plurality of outcome factors and, for each outcome factor that is modeled, the model comprising one or more respective outcome parameters of an implant system for a joint reconstruction of a joint of the patient; applying MoCap data of the patient's movement and geometric and inertial parameter data of the patient's movement and geometric and inertial parameter data for the patient to the model to generate an optimized position of the implant system for the patient, the optimized position generated by optimizing the one or more respective outcome parameters of at least two of the outcome factors; and providing the optimized position for presenting in association with a medical image comprising a bone of the patient associated with the joint.

There is also provided an apparatus such as a computing device comprising a processor and a storage device coupled to the processor and storing computer readable instructions that when executed by the processor configure the computing device to perform the methods according to the method embodiments.

### BRIEF DESCRIPTION OF THE DRAWINGS

FIG. 1 is an illustration of a network system comprising a plurality of respective computing devices, in accordance with an embodiment.

FIG. 2 is an illustration of a skeletal model, depicting example body segments for which geometric and inertial parameter data is provided to the KID model in accordance with an embodiment.

FIG. 3A is an illustration of an implant system in a THA in accordance with an embodiment.

FIG. 3B is an illustration of an implant impingement factor. FIG. 3C is an illustration of a bone-on-implant impingement factor. FIG. 3D is an illustration of an edge-loading factor.

FIGS. 4A and 4B are illustrations of an implant system position generated by a KID model module as a function of cup orientation (inclination and anteversion). FIG. 4A illustrates an implant system position generated by a KID model module by applying MoCap data for a sit-to-stand activity. FIG. 4B illustrates an implant system position generated by a KID model module by applying MoCap data for aggregated activities sit-to-stand, walking and picking up an object.

FIG. 5A and FIG. 5B are examples of a graphical user interface (GUI) for surgical planning, in accordance with embodiments, which GUI is presented by a surgical planning computing device of FIG. 1.

FIG. 6 is an illustration of data acquisition devices and data, specifically medical image data and MoCap data that may each have respective coordinate systems and may be coregistered for use in surgical planning, in accordance with an embodiment.

FIG. 7 is an illustration of data acquisition devices and data, specifically surgical navigation data and MoCap data that may each have respective coordinate systems and may be coregistered for use in surgical navigation, in accordance with an embodiment.

FIGS. 8 and 9 are flowcharts of respective operations of a computing device illustrating respective computer-implemented methods in accordance with embodiments.

It will be appreciated that for simplicity and clarity of illustration, elements shown in the figured have not necessarily been drawn to scale. For example, the dimensions of some of the elements may be exaggerated relative to other elements for clarity.

### DETAILED DESCRIPTION

High Level Overview

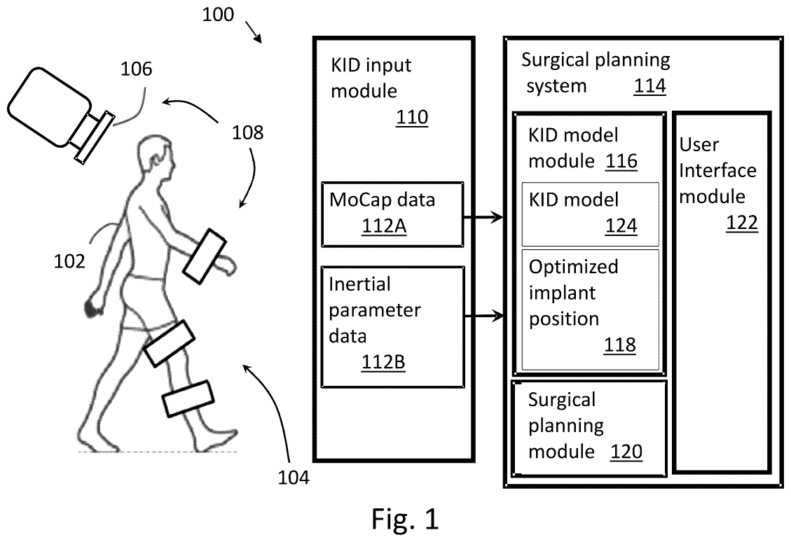

FIG. 1 is an illustration of a sensor-based network system 100 (e.g. abbreviated to system 100) for procedure planning such as for optimizing implant system positioning, in accordance with an embodiment. In an embodiment, a patient 102 is a candidate for a musculoskeletal surgery, in which the kinetic condition of the patient 102 is to be altered. For example, total joint arthroplasty (TJA) surgeries, such as hip and knee replacements, seek to improve the overall dynamic and kinetic condition of the patient through increased range of motion, pain free/reduced movement, and/or restoration of normal biomechanics.

The apparatus, methods and techniques described herein may be employed in any kind of anatomical reconstruction surgery. For the purpose of illustrating the invention, exemplary embodiments relating to total joint arthroplasty (TJA), and in some cases, THA, are described. Nevertheless, one skilled in the art will appreciate that the scope of the present disclosure extends to other types of anatomical reconstruction surgery, such as total knee arthroplasty (TKA) or shoulder replacement surgery, for example.

In an embodiment, system 100 is used to optimize the position of the implant system for the patient 102. MoCap data 112A is collected using a MoCap system 108 and provided to a KID input module 110. In accordance with an embodiment, the MoCap system 108 may use any type of motion capture technologies, such as optical marker-based motion capture, marker-less motion capture based on video feed, or inertial sensors and inertial measurement units (IMU), alone or in any combination, as further described below. The MoCap system 108 provides measurements of the motion (i.e. joint angles versus time and/or joint positions) of a patient's limbs during various activities, such as sit-to-stand, walking and picking up an object from the ground, for example.

In an embodiment, the MoCap system 108 may include one or more cameras 106 and/or one or more wearable optical and/or inertial devices 104 such as inertial sensors, inertial measurement units (IMUs), and/or passive or active optical markers, coupled to the patient's body 102 (e.g. their limb segments), as further described below.

The system 100 for optimizing implant system positioning may further comprise a plurality of computing devices comprising a KID input module 110 and a surgical planning system 114. In an embodiment, the KID input module 110 and the surgical planning system 114 may be implemented on a computer server or other computer device (not shown). The KID input module 110 may be implemented on a single computer device, or may be distributed amongst multiple devices. For example, the KID input module 110 functionality may be distributed amongst a dedicated server, or the surgical planning system 114. Other configurations may be used.

The KID input module 110 receives MoCap data 112A from the MoCap system 108. The MoCap system 108, may communicate with the KID input module 110 for example, using wired communication or wireless communication such as Bluetooth™ or WiFi.

In an embodiment, the KID input module 110 may receive MoCap data 112A as one or more separate datasets. Each MoCap data 112A dataset may represent the patient's motion for an individual activity. The KID input module 110 may process the one or more MoCap data 112A datasets to concatenate, aggregate or otherwise combine MoCap data 112A for one or more activities, as further described below. Alternately or in addition, the KID input module 110 may receive MoCap data 112A comprising the motion measured for one or more persons (not including the patient) performing one or more activities as one or more separate datasets, which may then be concatenated, aggregated or otherwise combined by the KID input module 110, as further described below. In an embodiment, the KID input module 110 may receive MoCap data 112A that has already been concatenated, aggregated or otherwise combined, as further described below.

The KID input module 110 may also receive geometric and inertial parameter data 112B, which comprises body segment lengths, body segment masses, body segment centers of mass, joint angles, and an inertia matrix (includes body segment moments of inertia and products of inertia), as further described below.

In an embodiment, the MoCap data 112A and the geometric and inertial parameter data 112B are processed to ensure data quality and standardization. For example, the KID input module 110 may perform data processing operations such as signal conditioning (e.g. low-pass filtering), and outlier rejection (e.g. implementing random sample consensus (RANSAC) algorithms). Further data processing may include converting the MoCap data 112A into a standardized data format such as comma-separated values (CSV) or JavaScript Object Notation (JSON).

The MoCap data 112A and the geometric and inertial parameter data 112B are provided by the KID input module 110 and are received by a KID model module 116 of a surgical planning system 114. The MoCap data 112A and the geometric and inertial parameter data 112B provided to the KID model module 116 may be a subset of that received by the KID input module 110. In an embodiment, the KID model module 116 may receive MoCap data 112A from the KID input module 110 as one or more separate datasets and may further process the MoCap data 112A to aggregate, concatenate or otherwise combine the separate datasets, as further described below.

The surgical planning system 114 may be implemented on a single computer device, or may be distributed amongst multiple devices, including on devices common to the KID input module 110. The KID model module 116 stores and uses a KID model 124 and performs computations on the MoCap data 112A and the geometric and inertial parameter data 112B to generate an optimized position of the implant system for the patient, as further described below. The KID model 124 models one or more outcome factors that represent physical phenomena which may affect the desired anatomical reconstruction of a joint of the patient. The outcome factors comprise one or more of an edge loading factor, an implant impingement factor, a bony impingement factor, a bone-on-implant impingement factor, and a soft tissue impingement factor. Each outcome factor is described further below.

In an embodiment, the outcome factors are associated with one or more outcome parameters of an implant system 300 for a joint reconstruction. The outcome parameters are variables used to quantify aspects of the implant system position with respect to the outcome factors. For example, an outcome factor involving impingement (e.g. implant impingement factor, bone-on-implant impingement factor, bony impingement factor and soft tissue impingement factor) may be quantified by an outcome parameter that quantifies the minimum angular or linear distance to impingement (i.e. minimum angular distance may mean the angle in the direction closest to impingement).

The skilled person will readily appreciate that the outcome factors may further comprise one or more other outcome parameters, such as limb length, or an angular or linear measure of the joint's range of motion.

The KID model module 116 receives the MoCap data 112A and the geometric and inertial parameter data 112B and uses the KID model 124 to generate an optimized implant system position 118 by optimizing the one or more outcome factors. In an embodiment, the optimized implant system position 118 comprises optimizing one or more of the outcome parameters associated with the one or more outcome factors by performing a combined optimization. In another embodiment, the optimized implant system position 118 comprises optimizing one or more outcome parameters associated with the one or more outcome factors while constraining one or more of the remaining outcome parameters associated with the one or more outcome factors. In this context, constrain means that the value of the constrained outcome factor should be maintained above a minimum threshold value, below a maximum threshold or within a range between an upper maximum and a lower minimum threshold. For example, in an embodiment, the edge loading factor may be mathematically optimized while maintaining the implant impingement factor and the bony impingement factor above a minimum threshold value.

With reference again to FIG. 1, a user interface module 122 is provided to enable a user (for example, a surgeon) to perform surgical planning, by providing information, visualizations, and controls for user interaction. The user interface module 122 may further allow the surgeon to select one or more outcome parameters for optimization or combined optimization. The surgical planning module 120 may also receive 2D or 3D medical images of the patient (i.e. of the anatomical structures to be altered or reconstructed during surgery). The surgical planning module 120 may provide spatial targets to the surgeon, via the user interface 122, based on the desired reconstruction. The spatial targets may be in the form of graphical overlays on medical images, for example, of implants, numerical information, graphical information, manipulations of medical images (for example, repositioning of selected image features, such as realigning pixels or voxels of a crooked bone to be straight), annotations, etc.

Motion Capture System and Data

The MoCap system 108 may use any available technology known to the skilled person to measure the motion of the patient while performing various activities such as sit-to-stand, walking, or picking up an object from the ground. In accordance with an embodiment, the MoCap system 108 may use any type of motion capture technologies, such as optical marker-based motion capture, optical marker-less motion capture based on video feed, or inertial sensors and inertial measurement units (IMU), alone or in combination.

The MoCap system 108 may comprise one or more cameras 106, including infrared cameras, visible spectrum cameras, and depth cameras (such as time of flight cameras), such as when using optical marker-based motion capture or marker-less motion capture based on video feed. In an embodiment, the one or more cameras 106 may capture a position and/or pose of the devices 104 attached to the patient 102.

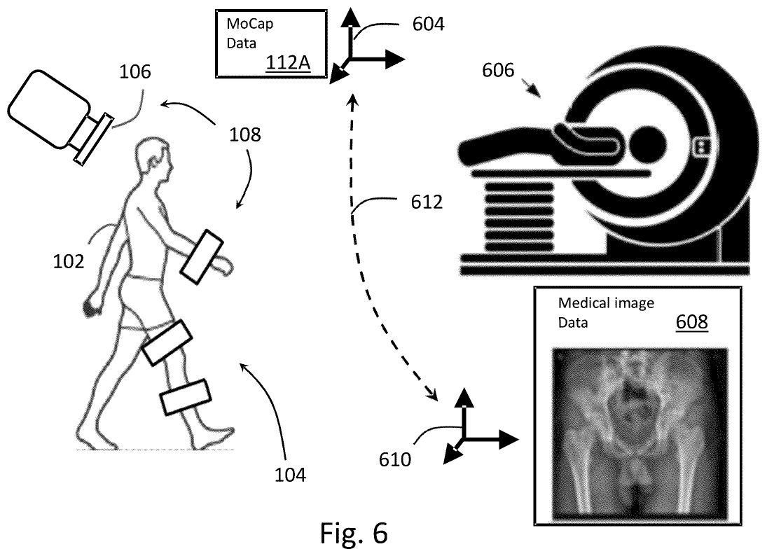

Optical marker-based motion capture uses cameras to track devices 104, such as optical devices (i.e. fiducials or fiducial markers) attached to the patient's body segments. The devices 104 may be aligned with specific bony landmarks or may have a known position relative to the MoCap data 112A coordinate system 604 (see FIG. 6), which is discussed below. In an embodiment, the patient 102 may wear a body suit with embedded or attached devices 104. In another embodiment, devices 104 such as optical marker arrays may be attached to the patient, comprising at least 3, preferably 4 or more individual optical markers (e.g. reflective spheres) arranged in a known pattern to capture the motion of the patient 102 while the patient performs one or more activities. The devices 104 may be passive or active optical devices. Passive optical markers may comprise reflective or retro-reflective features to reflect light, such as infrared light, back to one or more cameras 106, one or more of which may be an infrared camera. Active optical markers may emit light that can be detected by a camera 106. The devices 104 may further include radiopaque features for detection in x-ray images.

In an embodiment using optical marker-based motion capture, one or more cameras 106 may capture a series of sequential images to capture the position and/or pose of the devices 104 to measure the motion of a patient 102 while the patient (or another person) performs one or more activities.

In another embodiment using marker-less motion capture based on video feed, one or more cameras 106 may capture a series of sequential images to capture the position and/or pose of the patient's body segments to measure the motion of a patient 102 while the patient performs one or more activities. In the context of embodiments using marker-less motion capture based on video feed, the MoCap system 108 may not comprise devices 104. In another embodiment using marker-less motion capture based on video feed, the MoCap system 108 may comprise devices 104, such as inertial devices. The one or more devices 104 may include any type of inertial devices known in the art, such as accelerometers, gyroscopes, magnetometers, inertial measurement units (IMUs) and microelectromechanical system (MEMS) inertial sensors, alone or in any combination. In another embodiment, devices 104 may further comprise other sensors in addition or alternatively to the inertial devices, such as global positioning system (GPS) sensors and electromagnetic motion tracking sensors such as the Standard Sensor (Polhemus, Vermont, U.S.A.).

In yet another embodiment, the MoCap system 108 may not include one or more cameras 106, and the devices 104 may comprise inertial devices, IMUs, and/or other types of sensors as discussed above, in lieu of optical marker-based motion capture or marker-less motion capture based on video feed. The devices 104 may further include embedded radiopaque features for detection in x-ray images. In an embodiment, the embedded radiopaque features comprise at least 3, preferably 4 or more steel spheres with a known position relative to the MoCap data 112A coordinate system 604 (see FIG. 6).

The terms “sensor”, “marker” and “device” do not strictly mean a single sensor, marker or device, but may mean a collection of coupled sensors, markers and devices. Furthermore, the terms sensor, marker and device (whether used for an individual device, or multiple coupled devices) may include analog and/or digital devices for signal processing, data processing and transmission. For example, a Bluetooth (Bluetooth SIG, Inc., Kirkland, WA, U.S.A.) capable IMU may be considered a device, though it is comprised of 3 orthogonal accelerometers and gyroscopes, and on-board processing and radio devices to transmit the device data wirelessly using Bluetooth-based wireless communication.

In yet another embodiment, one or more cameras 106 may capture a position of the one or more devices 104 in combination with one or more inertial devices sensors, any of which may be attached to the patient.

The MoCap system 108 may comprise any combination of devices 104, including any of the aforementioned types of devices 104, including multiples of the same type of devices 104. The devices 104 may be used to measure a patient's motion during particular activities by being coupled to anatomical structures of the patient 102 during movement (e.g. IMU sensors may be strapped to a patient's limb segments while they are performing a prescribed motion). The devices 104 may measure the patient's motion without patient contact, for example, optically. A camera 106 may be configured to measure the pose of a patient (conducting a prescribed motion) within its field of view. The pose of the patient may include the poses of each individual relevant body segment of the patient. The above examples demonstrate that there are many ways in which devices 104 may be used to measure the motion of a patient, and this description considers all possible ways of measuring a patient's motion without limitation to specific technologies or implementations.

The MoCap data 112A may be collected for a single activity or for multiple activities (i.e. measured while the patient undertakes one or more activities). The activities may be routine daily activities, such as walking, running, riding a bicycle, sitting down, standing up, putting on a shoe or bending to pick up an object. Alternatively or in addition, the activities may be selected based on the characteristics of the patient, such as the patient's age, hobbies (e.g. sporting activities such as swinging a golf club or swimming), line of work, medical history, etc. For example, for a patient who plays hockey, MoCap data 112A may be collected while the patient plays or simulates playing hockey.

Alternatively or in addition to the data collected of the patient performing one or more activities, MoCap data 112A may be collected from one or more persons (i.e. who are not the patient), and/or obtained from a private or public database for one or more persons (i.e. who are not the patient) performing one or more activities. For example, MoCap data 112A could be obtained from a public database, such as the Carnegie Melon University's Graphics Lab MoCap dataset (publicly available database) or a publicly-available patient database. MoCap data 112A may also be obtained from a private patient database.

The MoCap data 112A (regardless of whether it was obtained from the patient, other persons or a private or public database) may be provided to the KID input module 110 as one or more separate datasets and the one or more separate datasets may represent MoCap data 112A for a single patient or other person performing a single activity or may represent data from two or more persons, one of which may be the patient, performing one or more activities. The MoCap data 112A may be concatenated, aggregated or otherwise combined for one or more of the patient or other persons performing one or more activities by the KID input module 110 and/or the KID model module 116. Alternatively, the MoCap data 112A may already be combined prior to being received by the KID input module 112A. The combination or aggregation of MoCap data 112A may include calculating one or more statistics of the motion, such as an average, a maximum, a minimum and/or a percentile (i.e. a 90th percentile, a 50th percentile, a 25th percentile, etc). The one or more persons may have certain characteristics in common with the patient, such as age, body type, occupation, gender, joint pathology, or may participate in the same types of activities (i.e. sporting activities).

It is preferred that at least some of the MoCap data 112A applied to the KID model 124 is patient-specific, for example, collected from the patient by the MoCap system 108. However, MoCap data 112A collected from one or more persons instead of or in addition to the patient may also be patient-specific, such as when the one or more persons has certain characteristics in common with the patient. The skilled person will appreciate that the characteristics of interest and the degree of similarity between the patient and the one or more persons will vary with the objectives of the surgery, the type of surgery, and the characteristics of the patient.

The MoCap data 112A collected for one or more activities may be applied to the KID model 124 as an individual activity. Alternatively or in addition, the MoCap data 112A for two or more activities may be aggregated, concatenated or otherwise combined and applied to the KID model 124 as an aggregated motion representing the motion from two or more activities, as further discussed below.

Geometric and Inertial Parameter Data

Geometric and inertial parameter data 112B refers to the properties of body segments that are related to the geometry of the body segments, such as body segment lengths, or the inertial properties of the body segments, such as the body segment masses, body segment centers of mass, and the inertia matrix. Inertial parameter data is related to but different from inertial sensors and inertial measurement units. In the context of this disclosure, inertial parameter data refers to the properties of the body segments related to the inertia of the body segments. Inertial sensors and inertial measurement units may be used to measure the motion of the body segments of the patient, as discussed previously.

The geometric and inertial parameter data 112B may be obtained from medical imaging, cadaver measurements, publicly available databases or predictive equations known in the art. Some geometric and inertial parameter data 112B may be measured directly from the patient, such as body segment lengths.

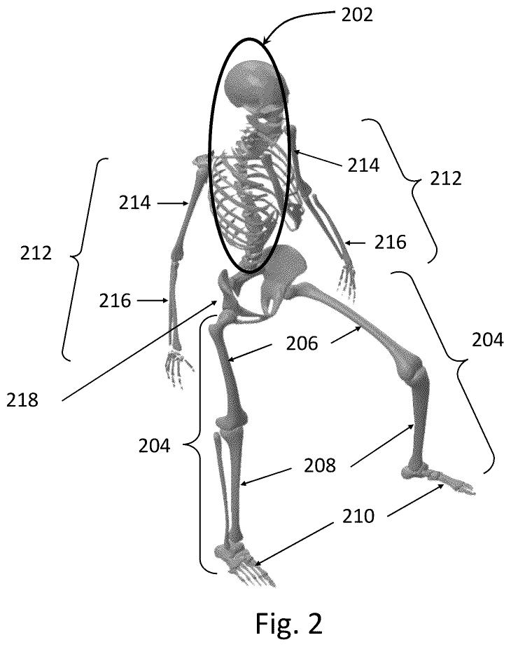

FIG. 2 depicts example body segments for which geometric and inertial parameter data 112B is received by the KID input module 110 and provided to the KID model module 116. The geometric and inertial parameter data 112B may be associated with individual body segments or collective body segments of the same patient or person (e.g. the head and trunk of the patient may be combined into a collective body segment or may be treated as individual body segments). In an embodiment, the geometric and inertial parameter data 112B comprises: the trunk 202 of the patient (which includes the patient's head), a thigh 206 for each of the patient's two legs 204, a shank 208 for each of the patient's two legs 204, a foot 210 for each of the patient's two legs 204, an upper arm 214 for each of the patient's two arms 212 and a forearm 216 for each of the patient's two arms 212, and a pelvis 218.

It will be apparent to the person skilled in the art that the geometric and inertial parameter data 112B may comprise any combination of body segments, depending on the specifics of the patient, the type of joint replacement surgery, the objectives of the joint replacement surgery and the activities for which MoCap data 112A is applied to the KID model 124. For example, the trunk 202 may be divided into smaller body segments with joints between them. In an embodiment, the head and neck may be represented by two separate body segments. This approach may be employed when, for example, the motion of the head and neck are expected to influence the optimized implant system position 118 generated by the KID model 124. In another embodiment, the geometric and inertial parameter data 112B may comprise a subset of the aforementioned body segments, such as one or more of a trunk 202, a thigh 206 for each of the patient's two legs 204, a shank 208 for each of the patient's two legs 204, a foot 210 for each of the patient's two legs 204, and a pelvis 218.

The Implant System

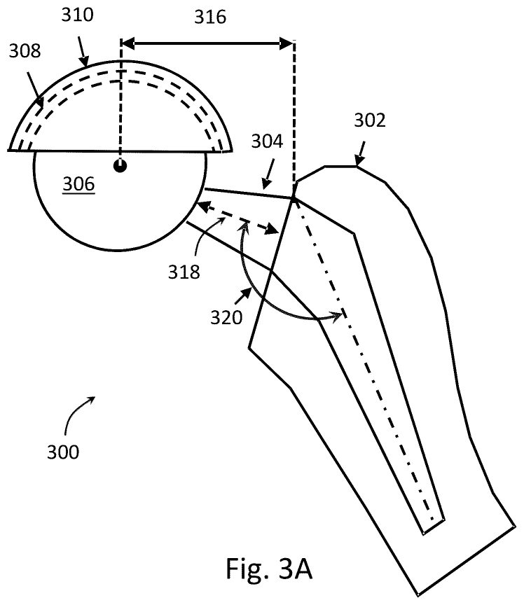

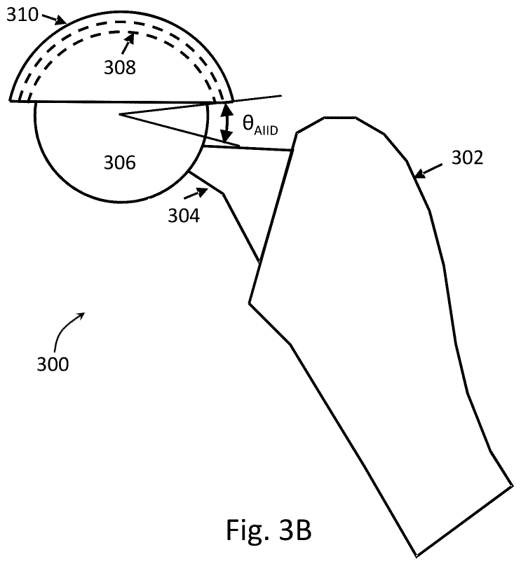

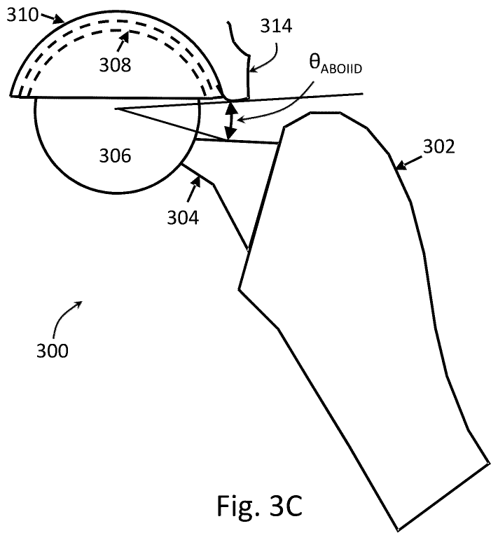

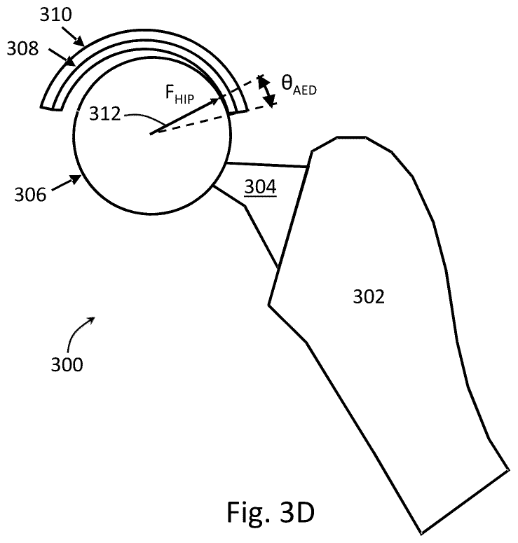

The implant system is an artificial joint. FIG. 3A depicts the implant system 300 for an artificial hip joint in accordance with an embodiment. The implant system 300 comprises a cup 310, a liner 308, a femoral head 306, and a femoral stem 304. The femoral head 306 is rigidly attached to the femoral stem 304, which is rigidly attached to the femur 302 of the patient's leg 204. The liner 308 is received in and rigidly fixed to the cup 310. In the context of this description, the liner 308 and cup 310 are positioned as a single unit (i.e. the orientation or translation of the cup 308 implies the orientation or the translation of the liner 308, and vice versa). The femoral head 306 is received in the liner 308 and is free to rotate within the liner 308.

The implant system position is associated with one or more of: a cup orientation (rotation of the cup 310 and the associated liner 308, comprising cup inclination and cup anteversion); a translational cup position (translation of the cup 310 and liner 308 in any linear direction); a femoral version (rotation of the femoral stem 304 which is rigidly fixed to the patient's femur 302); a femoral head size (i.e. a diameter of the femoral head); a femoral stem size, which includes a stem neck length 318; a femoral stem offset 316 (shown in FIG. 3A as a medial stem offset); and a femoral neck-shaft angle 320. The one or more aspects of the implant system position may be optimized to generate an optimized implant position 118 of the patient 102.

The femoral implant depicted in FIG. 3A is visually different from that depicted in FIGS. 3B-3D, primarily because the portion of the stem 304 inserted into the femur is not shown in FIGS. 3B-3D. However, the various implant parameters discussed in relation to any of FIG. 3A-3D apply to all variations of femoral implants, including all those depicted and described herein.

Outcome Factors and Parameters

The KID model 124 models one or more outcome factors. The outcome factors comprise any of an edge loading factor, an implant impingement factor, a bony impingement factor, a bone-on-implant impingement factor and a soft tissue impingement factor. The KID model 124 comprises one or more outcome parameters of an implant system for the one or more outcome factors, which are variables employed by the KID model 124 to quantify one or more aspects of the position of the implant system 300 of the patient 102. For example, one or more outcome parameters may indicate the angle before impingement between two or more implant components (e.g. the cup 310 and the stem 304) or between two bones of the hip joint or adjacent to the hip joint (e.g. the femur 306 and the pelvis). In another example, one or more outcome parameters may indicate the linear distance before impingement between two or more bones of the hip joint or adjacent to the hip joint or between soft tissue and the implant system.

FIG. 3B depicts the implant impingement factor. The implant impingement factor accounts for potential contact between the femoral stem 304 and the acetabular cup 310, which can reduce the range of motion of the hip joint and/or cause pain or discomfort. During certain activities performed by the patient, the femoral head 304 may be leveraged out of the cup 310, resulting in dislocation of the artificial hip. The implant impingement factor may comprise one or more outcome parameters, such as an angular implant impingement distance, θ_{AIID} (AIID) The AIID is the angular distance between the femoral stem 304 and the edge of the cup 310 before impingement occurs for a particular position of the implant system 300. The femoral head 306 is rigidly attached to the femoral stem 304, and the femoral stem 304 is rigidly attached to the femur 302. During performance of an activity, such as walking, the motion of the patient's leg 204 (e.g. in flexion, extension, abduction, adduction) causes the femoral head 306 to rotate within the cup 310 and liner 308, altering the spatial relationship between the implant system 300 and the anatomy of the patient. As the femoral head 306 and the rigidly attached femoral stem 304 rotate counter-clockwise (on the page in accordance with the depiction in FIG. 3B), the AIID decreases. In the embodiment depicted in FIG. 3B, a positive AIID value (e.g. θ_{AIID}>0) indicates that impingement has not occurred between the femoral stem 304 and the cup 310 and/or liner 308. An AIID value of zero (e.g. θ_{AIID}=0) or a negative AIID value (e.g. θ_{AIID} <0) means that impingement has occurred between the femoral stem 304 and the cup 310 and/or liner 308. It will be apparent to one skilled in the art that the AIID may be defined differently such that a different threshold may define when impingement has occurred.

The bony impingement factor accounts for potential contact between two or more bony structures of the hip joint and/or adjacent to the hip joint, such as between the femur 302 and the pelvis. Contact between the bony structures can limit range of motion, cause pain or discomfort for the patient, and in some cases, cause the femoral head 306 to be leveraged out of the acetabular cup 310 and liner 308, resulting in hip dislocation. The bony impingement factor may comprise one or more outcome parameters. In an embodiment, the outcome parameters include the angular bony impingement distance, θ_{ABID} (ABID), which may represent the angular distance between two or more of the bony structures of the artificial hip joint and/or adjacent to the artificial hip joint (e.g. the femur 302 and the pelvis) for a particular position of the implant system 300. For example, the ABID may represent the angular distance between the lesser trochanter of the femur 302 and the ischium of the pelvis. The outcome parameters associated with the bony impingement factor may also include the linear bony impingement distance (LBID), which is the linear distance between at least two bony structures of the artificial hip joint and/or adjacent to the artificial hip joint.

While performing certain activities, such as walking, the motion of the patient's leg (e.g. extension, flexion, abduction or adduction) may alter the spatial relationship between the implant system 300 and the anatomy of the patient. In an example, the position of the implant system may result in impingement between the femur 302 (i.e. the lesser trochanter of the femur 302) and the pelvis (i.e. the ischium). The ABID and/or the LBID may provide an indication of the angular or linear range of motion of the artificial joint before bony impingement occurs. In an embodiment, a positive ABID (e.g. θ_{ABID}>0) and/or a positive LBID (e.g. LBID>0) means bony impingement has not occurred, whereas a value of zero or a negative ABID (e.g. θ_{ABID}<0) or LBID (e.g. LBID<0) means that bony impingement has occurred. It will be apparent to one skilled in the art that the ABID and LBID may be defined differently such that a different threshold may define when impingement has occurred.

During certain activities, the position of the implant system 300 may also result in impingement between bony structures of the hip joint and/or adjacent to the hip joint and the implant system 300, which may be accounted for by the bone-on-implant impingement factor. For example, there can be impingement between the pelvis and the femoral stem 304 or the femur 302 and the cup 310. Bone-on-implant impingement can reduce range of motion, cause pain or discomfort for the patient, and in some cases, may result in the femoral head being leveraged out of the acetabular cup. The bone-on-implant impingement factor is illustrated in FIG. 3C and may comprise one or more outcome parameters. In an embodiment, the outcome parameters include the angular bone-on-implant impingement distance, θ_{ABOIID} (ABOIID), which represents the angle between bony structures of the hip joint and/or adjacent to the hip joint and the implant system 300 for a particular position of the implant system 300. For example, the ABOIID may quantify the angular distance between the femoral stem 304 and the bony structure of the acetabulum 314 before impingement occurs.

The femoral head 306 is rigidly attached to the femoral stem 304, which is rigidly attached to the femur 302. Therefore, as the patient moves their leg 204 during certain activities (i.e. in flexion, extension, abduction, adduction), the femoral head 306 rotates within the cup 310 and liner 308. As the femoral head 306 and the rigidly attached femoral stem 304 rotate counter-clockwise (on the page in accordance with the depiction in FIG. 3C), the femoral stem 304 may impinge the bony structure of the acetabulum 314. In an embodiment in accordance with FIG. 3C, a positive ABOIID value (e.g. θ_{ABOIID}>0) means the femoral stem 304 has not impinged bony structure of the acetabulum 314. A value of zero or a negative ABOIID value (e.g. θ_{ABOIID}<0) means that the femoral stem 304 has impinged the bony structure of the acetabulum 314. It will be apparent to one skilled in the art that bone-on-implant impingement is not limited to impingement between a bony structure of the acetabulum 314 and the femoral stem 304, but could also occur between the femur 302 and the cup 310 and/or liner 308. For example, if the stem offset 316 (shown in FIG. 3A) were reduced. It will also be apparent to one skilled in the art that the ABOIID may be defined differently such that a different threshold may define when impingement has occurred.

In addition to bony structures, a hip joint also comprises soft tissue, such as muscles, ligaments, the labrum and the joint capsule. These tissues serve to stabilize the joint and control joint movement. However, soft tissues can also restrict range of motion of the hip joint and during certain poses and/or motions, the positioning of the implant system can result in impingement between the implant system and the soft tissues and/or the bony structures of the hip joint and the soft tissues. As a result, the patient may experience pain and/or reduced range of motion. The soft tissue impingement factor may comprise one or more outcome parameters. In an embodiment, the outcome parameters include the angular soft tissue impingement distance, OASTID (ASTID), which represents the angle between at least one soft tissue element and a bony structure of the hip joint and/or adjacent to the hip joint and/or between at least one soft tissue element and the implant system 300, for a particular position of the implant system 300. The outcome parameters may further include the linear soft tissue impingement distance (LSTID), which represents the linear distance between at least one soft tissue element and a bony structure of the hip joint and/or adjacent to the hip joint and/or between at least one soft tissue element and the implant system 300 for a particular position of the implant system.

For example, while performing certain activities, such as walking, the motion of the patient's leg (e.g. extension, flexion, abduction or adduction) may alter the spatial relationship between the implant system 300 and the anatomy of the patient. Depending on the position of the implant system 300, when the motion of the patient 102 causes the femoral head 306 to rotate, the altered position of the implant system may result in impingement between a soft tissue element and a bony structure of the hip joint and/or adjacent to the hip joint or between a soft tissue element and an element of the implant system 300. For example, the position of the implant system 300 may result in impingement between the labrum and the femur 302 (i.e. the greater trochanter of the femur 302). One skilled in the art will appreciate that impingement may occur, instead or in in addition, between a different soft tissue element, such as the joint capsule, and the implant system 300 (e.g. the stem 304). In an embodiment, a positive ASTID (e.g. θ_{ASTID}>0) and/or a positive LSTID (e.g. LSTID>0) means soft tissue impingement has not occurred, whereas a value of zero or a negative ASTID (e.g. θ_{ASTID}<0) or LSTID (e.g. LSTID<0) means that soft tissue impingement has occurred. It will be apparent to one skilled in the art that the ASTID and LSTID may be defined differently such that a different threshold may define when impingement has occurred.

The edge loading factor is depicted in FIG. 3D and refers to the condition that occurs when the contact force between the femoral head 306 and the cup 310 (i.e. the hip contact force 312) is located proximal to the edge of the cup 310. Edge-loading can result in premature and/or accelerated wear of the cup 310 and/or liner 308, specifically the liner 308. Further, when the hip contact force 312 is not directionally aligned to direct the femoral head towards the acetabular cup 308, edge loading may cause the femoral head 306 to separate from the acetabular cup 308, resulting in hip dislocation.

In an embodiment, the hip contact force 312 quantifies the magnitude and angle of the force applied by the femoral head 306 on the cup 310 and liner 308, as further described below. The edge loading factor may comprise the outcome parameter, the angular edge-loading distance, θ_{AED} (AED). The AED defines the angle between the direction of the hip contact force 312 and the edge of the cup 310 (and the liner 308). The femoral head 306 is rigidly attached to the femoral stem 304, which is rigidly attached to the femur 302. Therefore, as the patient moves their leg 204 (i.e. in flexion, extension, abduction, adduction), the motion causes the femoral head 306 to rotate within the liner 308 and cup 310. As the femoral head 306 rotates clockwise (as shown on the page in FIG. 3D), the angle of the hip contact force 312 may also rotate clockwise. When the hip contact force 312 points towards the edge of the cup 310, the magnitude of the hip contact force 312 is concentrated on the edge of the cup 310, which can cause the liner 308 to wear. As the hip contact force 312 rotates to a position below the edge of the cup 310 and liner 308, the hip contact force 312 may cause the femoral head 306 to separate from the cup 310 and liner 308, which may result in hip dislocation. The greater the angular edge-loading distance, the greater the range of motion of the implant system of the patient before edge-loading occurs.

A desired outcome of THA is to maximize the range of motion of the implant system 300 of the patient 102 for which one or more of the AIID, ABID, LBID, ABOIID, ASTID, LSTID and/or AED remain above a minimum acceptable threshold (as defined within the embodiments disclosed herein) while the patient performs one or more activities. One skilled in the art will appreciate that one or more outcome parameters may further be defined to have a maximum threshold, such that maximizing the range of motion of the implant system 300 involves one or more outcome parameters remaining above a minimum threshold while simultaneously remaining below a maximum threshold. In other embodiments, the one or more outcome parameters may be defined such that maximizing the range of motion of the implant system 300 requires one or more outcome parameters to remain below a minimum threshold.

One or more aspects of the implant system position 118 may influence the value of one or more outcome parameters, such as the AIID, ABID, LBID, ABOIID, ASTID, LSTID and/or AED, each of which may influence the range of motion of the implant system 300 of the patient 104 while the patient performs one or more activities. More specifically, one or more of the translational cup placement, the cup orientation, the femoral version, the femoral head size, the cup size (which includes the liner size), the stem size (which includes the stem neck length 318), the stem offset 316, and the femoral neck-shaft angle 320 may be optimized to optimize the value of one or more outcome parameters in accordance with the MoCap data 112A.

For example, in accordance with FIG. 3B, rotating the cup 310 and liner 308 clockwise reduces the AIID compared to the position of the implant system 300 depicted in FIG. 3B, whereas rotating the cup 310 and liner 308 counter-clockwise increases the AIID compared to the position of the implant system 300 depicted in FIG. 3B. However, in accordance with FIG. 3D, the same cup 310 and liner 308 rotation may have the opposite effect on the value of AED For example, rotating the cup 310 and liner 308 clockwise increases the AED compared to the position of the implant system 300 depicted in FIG. 3D and rotating the cup 310 and liner 308 counter-clockwise reduces the AED compared to the position of the implant system 300 depicted in FIG. 3D.

In another example, reducing the stem offset 316 (i.e. the medial stem offset as shown in FIG. 3A) may reduce the LBID, reducing the range of motion of the implant system 300 of the patient 102 during certain activities before bony impingement occurs. Similarly, reducing the stem offset 316 may reduce the ABOIID and therefore the range of motion of the implant system 300 of the patient 102 before bone-on-implant impingement occurs. In another example, altering the translational cup placement may similarly alter the LBID and/or the ABID, altering the range of motion of the implant system 300 of the patient 102 before bone-on-implant impingement occurs. In yet another example, altering the femoral version (the angle of the stem 304 with respect to the femur 302) may alter the ABOIID, the ABID or the ASTID. Altering the femoral version may reduce the range of motion of the implant system 300 of the patient 102 while the patient performs certain activities, before bone-on-implant impingement or bony impingement occurs. A person skilled in the art will appreciate that various aspects of the implant system position may affect one or more of the outcome parameters.

Kinematic and Inverse Dynamic (KID) Model

In accordance with an embodiment, the KID model module 116 may be implemented as computer instructions to execute on a computing device (e.g. a device of surgical planning system 114). The KID model module 116 applies the MoCap data 112A and the geometric and inertial parameter data 112B to the stored KID model 124 to generate an optimized implant system position 118 for the implant system 300 (shown in FIG. 3A). The KID model module 116 further generates an optimized position of the implant system 300 for the patient in accordance with the MoCap data 112A by optimizing the one or more respective outcome parameters of at least one of the outcome factors for at least one aspect of the position of the implant system 300.

The computer model may be implemented as a physics-based model based on the kinematics and inverse dynamics of human body movement. For example, the computer model may be implemented as a parameterized kinematic and inverse dynamic model of a human body (parameters such as weight, length, width, height, strength, moments of inertia, centers of mass, joint angles, joint moments, joint torques and joint forces, etc. may be associated with individual or collective body segments). The computer model may determine pre-operative and patient specific computer models based on applying MoCap data 112A and geometric and inertial parameter data 112B, at least some of which is specific to the patient. The computer model may also determine expected post-operative patient specific computer models based on applying the MoCap data 112A, the geometric and inertial parameter data 112B and one or more specific instances of the implant position. For instance, the computer model may apply the MoCap data 112A and the geometric and inertial parameter data 112B to the KID model 124 to estimate the joint forces (i.e. the hip contact force), joint angles, the body segment angles and positions and/or poses, the moments and forces generated by the joints based on the motion of the joints (i.e. joint angle through time), which may be used alone or in combination by the model to model one or more outcome factors to generate the optimized implant system position 118.

In an embodiment, the KID model 124 is a musculoskeletal model. The musculoskeletal model comprises body segments as shown in FIG. 2 and discussed above and degrees of freedom are defined for each joint. The KID model 124 further comprises a known muscle geometry model such as the OpenSim model discussed by A. Rajagopal et al, in “Full-body musculoskeletal model for muscle-driven simulation of human gait”, published in 2016 in IEEE Transactions on Biomedical Engineering Volume 63 at pages 2068-2079 (Rajagopal), incorporated herein by reference. The MoCap data 112A and the geometric and inertial parameter data 112B are applied to the KID model 124 to calculate the hip contact force 312 and the position of the body segments (e.g. femur 302, pelvis) connected to the implant system 300 of the artificial hip joint at each instant of the motion (i.e. the motion defined by the MoCap data 112A). The KID model 124 may also generate estimated ground forces and moments exerted on each of the patient's (two) feet without using force plate data. The KID model 124 may model one or more outcome factors to generate an optimized implant system position 118 by optimizing one or more outcome parameters in accordance with the MoCap data 112A and the geometric and inertial parameter data 112B.

In an embodiment, the KID model 124 comprises and is assumed to be actuated by joint torques for all joints except the pelvis joint connecting the pelvis to the ground frame. The KID model 124 further comprises the resultant of ground reaction forces, applied in the KID model 124 as external forces and moments on each foot 210. The KID model 124 comprises the equation:

M(q)\ddot{q}+C(q,\dot{q})\dot{q}+G(q)=Q(q)[F_{G}τ] (1)

Where q is the generalized coordinates, M (q) is the mass matrix for the body segments (i.e. geometric and inertial parameter data 112B provided to the KID model module 116), C (q, \dot{q}) contains the Coriolis forces, G(q) includes the gravity forces, τ is the vector of the joint torques and F_{G} contains the ground reaction forces and moments exerted on each foot 210. The variables q, \dot{q} and \ddot{q} represent the MoCap data 112A applied to the KID model module 116, where \dot{q} and \ddot{q} represent the first and second derivatives of q. Q (q) is a function that maps the ground reaction forces and moments as well as joint torques into joint space. The KID model 124 applies the MoCap data 112A and the geometric and inertial parameter data 112B and performs inverse dynamic analysis to estimate the joint torques. In an embodiment, the KID model performs an estimation of the ground reaction forces and moments, eliminating the need for force plate measurements. When the patient 102 is supported by one foot, for example, when the patient is walking, the KID model 124 performs an estimation of the joint torques and the reaction forces and moments by setting the ground reaction forces and moments on the airborne foot to zero, and in accordance with the MoCap data 112A and the geometric and inertial parameter data 112B. The KID model 124 computes the remaining ground reaction forces, moments and torques in accordance with Eq. (1). When both feet are in contact with the ground, force plate measurements may be used to determine ground reaction forces and moments. However, force plate measurements are not practical or cost efficient. In an embodiment, when the patient's feet 210 are both in contact with the ground, the KID model 124 performs an estimation of the joint torques and the reaction forces and moments using kinematics and dynamical properties, such as using the method proposed by S. Skals et al, in “Prediction of ground reaction forces and moments during sports-related movement” published in 2017 in Multi-body System Dynamics Volume 39 at pages 175-195, which is incorporated herein by reference.

For example, in an embodiment, the KID model 124 estimates the moment of the resultant ground reaction forces about the center of the ankle for the case of no slipping of the foot on the ground by solving the second-order quadratic equation optimization problem given in Eq. (2), in accordance with Eq. (1):

$${min}_{τ , {F}_{G}} J ( τ , {F}_{G} ) = {{F}_{G}}^{T} {SF}_{G} + {\sum }_{i = 1}^{23} {( \frac{{τ}_{i}}{{τ}_{i , max}} )}^{2} \qquad ( 2 )$$

In Eq. (2), S is any positive definite matrix.

The KID model 124 may perform computations to estimate muscle forces using static optimization techniques and force equilibrium techniques well-known to the skilled person. For example, in an embodiment, the KID model 124 performs computations to estimate the hip contact force 312. The KID model 124 may comprise 18 muscles around the hip joint. The skilled person will appreciate that fewer or more muscles may be included to model the hip forces. In an embodiment, muscle geometry, origin/insertion points and wrapping geometries may be obtained from an OpenSim model (such as that disclosed by Rajagopal et al, discussed above) and the muscle elements may be modeled with a simple muscle model without contraction/activation dynamics. In the embodiment, the KID model 124 may estimate muscle forces by solving the muscle recruitment problem through static optimization, for example, by minimizing the sum of the cubed muscle forces normalized by the strength of the muscle. The embodiment may further comprise optimization constraints, such as ensuring that the muscles can only be in tension. The KID model 124 may estimate the hip contact force 312 from the muscle forces as the hip joint reaction force using known force equilibrium techniques.

The KID model module 116 applies the MoCap data 112A and the geometric and inertial parameter data 112B to the KID model 124 to model the outcome factors (discussed above) to generate an optimized implant system position 118 by optimizing one or more outcome parameters, as further discussed below.

Optimization

The KID model module 116 applies the MoCap data 112A and the geometric and inertial parameter data 112B to the stored KID model 124 to generate an optimized implant system position 118 for the implant system 300. The position of the implant system 300 is associated with one or more of a cup orientation, a translational cup position, a femoral version, a femoral stem size, including neck length 318, and a femoral stem offset 316. Changing one or more of the elements associated with the position of the implant system 300 may change the values of one or more outcome parameters associated with one or more outcome factors. For example, changing a cup orientation may change a value of one or more of AIID, ABID, ABOIID, ASTID, and/or AED. Similarly, changing a femoral stem offset 316 may change one or more of ABID, LBID, ABOIID, LBID, ASTID, LSTID and/or AED. Other combinations of variations in the elements of the position of the implant system 300 may change the values for one or more of the outcome parameters associated with the one or more outcome factors. Therefore, the KID model module 116 optimizes the position of the implant system 300 for the patient in accordance with the MoCap data 112A by optimizing the one or more respective outcome parameters of at least one of the outcome factors for at least one aspect of the position of the implant system 300.

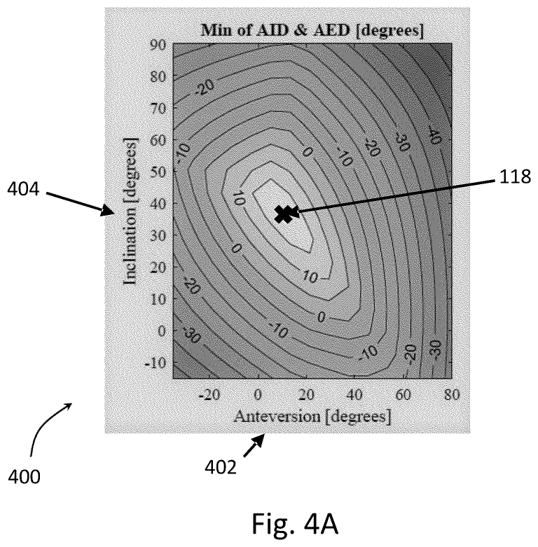

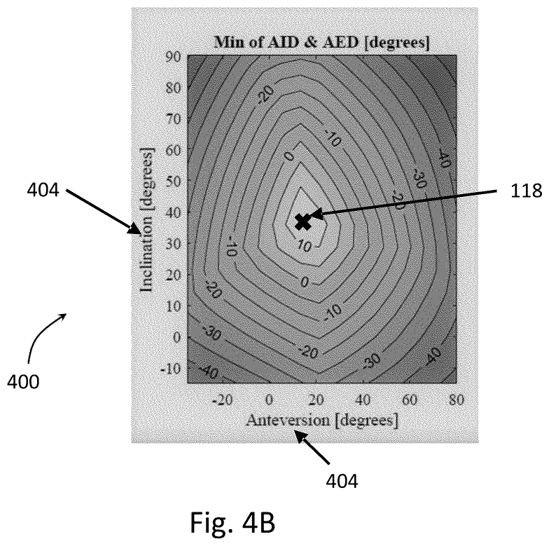

In an embodiment, the KID model module 116 optimizes the position of the implant system by performing a combined optimization of two or more outcome parameters of the two or more outcome factors. For example, in one embodiment, the AIID and the AED may be optimized by performing a combined optimization of the position of the implant system. As discussed above, rotating the cup 310 and liner 308 counter-clockwise (as shown on the page in FIG. 3B) increases the AIID However, rotating the cup 310 and liner 308 counter-clockwise (as shown on the page in FIG. 3B or 3D) decreases the AED. Similarly, orienting the cup 310 and liner 308 to increase the AED, for example, by rotating the cup 310 and liner 308 clockwise decreases the AIID The KID model module 116 may perform a combined optimization to maximize the minimum value of the AIID and the AED simultaneously in accordance with the MoCap data 116 for one or more activities. FIGS. 4A and 4B illustrate the combined optimization for the AED and AIID using MoCap data 112A collected for the activity “sit-to-stand” and MoCap data 112A aggregated for the three activities “walking”, “sit-to-stand”, and “picking up an object”, respectively.

FIG. 4A illustrates example results of a combined optimization of the AED and the AIID generated by the KID model module 116 when MoCap data 112A for the activity “sit-to-stand” is applied. The minimum value of the AIID and the AED is shown as a function of the cup orientation (anteversion 402 and inclination 404) aspect of the position of the implant system 300. Positive values represent cup orientations for which neither implant impingement nor edge-loading occurred in accordance with the MoCap data 112A (i.e. which represents sit-to-stand in this example). As shown in FIG. 4A, the optimized implant system position 118 generated by the KID model module 116 in this example comprises an anteversion of 7° and inclination of 39°. The corresponding maximum of the minimum values of both the AIID and the AED in accordance with the MoCap data 112A is 19° (as indicated by the contours shown in the example results in FIG. 4A for the optimized implant position 118). MoCap data 112A collected while a specific patient performs other activities (relative to sit-to-stand), such as swinging a golf club, running, swinging a baseball bat, climbing stairs, swimming, etc., may be applied to the KID model 124 instead of or in addition to the MoCap data 112A for the activity sit-to-stand to generate an optimized implant system position 118 of the implant system 300. Alternatively or in addition, the MoCap data 112A applied to the KID model 124 may include data collected from one or more persons that are not the patient, as discussed above. may be collected for one or more persons performing one or more activities and aggregated, as discussed above.

FIG. 4B illustrates the results of a combined optimization of the AIID and the AED using MoCap data 112A aggregated for three activities: walking, sit-to-stand and picking up an object. The minimum value of the AIID and the AED are shown as a function of the cup orientation (anteversion angle 402 and inclination angle 404) aspect of the position of the implant system 300. Positive values represent cup orientations for which neither implant impingement nor edge-loading occurred during the three activities (sit-to-stand, walking and picking up an object) in accordance with MoCap data 112A aggregated into a single MoCap data set. As shown in FIG. 4B, the optimal implant system position 118 generated by the KID model module 116 is an anteversion of 16° and inclination of 36°. The corresponding maximum of the minimum values of both the AIID and the AED is 18° (as indicated by the contours shown in the example results in FIG. 4B for the optimized implant position 118). Instead of or in addition, MoCap data 112A collected while a specific patient performs other activities (relative to sit-to-stand, walking and picking up an object) may be aggregated and applied to the KID model 124. Other activities may include swinging a golf club, running, swinging a baseball bat, climbing stairs, swimming, etc. Alternatively or in addition, the MoCap data 112A applied to the KID model 124 may include data collected from one or more persons that are not the patient, as discussed above.

The person skilled in the art will readily appreciate that the combined optimization performed by the KID model module 116 may be a combined optimization for any of the outcome parameters of the one or more outcome factors discussed herein. For example, the combined optimization may include two, three, four or five of the outcome parameters discussed herein. The preferred selection of outcome parameters in the combined optimization depends on the specific characteristics of the patient and the specific objectives of the surgery. The skilled person will further appreciate that altering one or more of a translational cup position, a femoral version, a femoral stem size or a femoral stem offset may affect one or more outcome parameters of the one or more outcome factors. Therefore, the skilled person will appreciate that the combined optimizations performed by the KID model module 116 may comprise any number of outcome parameters and any number of elements of the implant system 300.

In another embodiment, the KID model module 116 may generate the optimized implant system position 118 by performing an optimization of one or more outcome parameters of the one or more outcome factors while constraining one or more of the remaining outcome parameters. For example, in this embodiment, the optimized implant system position 118 may comprise maximizing the minimum value of the AIID in accordance with the MoCap data 112A for one or more activities while constraining the AED to remain above a minimum threshold. The skilled person will appreciate that two or more outcome parameters, in any combination, may be optimized or constrained to be above a minimum threshold by the KID model module 116 to generate the optimized implant position 118. Further, the optimized implant system position may include one or more of a cup orientation, a cup translational position, a femoral version, a femoral stem size and a femoral stem offset.

In accordance with an embodiment, the KID model module 116 generates the optimized implant system position 118 by implementing optimization operations, for example, using linear, non-linear, or integer optimization techniques. Clinically and physically relevant cost functions and constraints may be implemented in the optimization operations. For example, an optimization cost function may include minimizing a Euclidean norm associated with boney impingement or implant impingement for a total hip arthroplasty. A cost function may include minimizing edge loading of implants, to prevent premature wear or risk of dislocation, or to maximize range of motion before implant impingement, bony impingement, bone-on-implant impingement or soft tissue impingement occurs.

In an embodiment, the cost function may include setting higher or lower thresholds on different outcome parameters to constrain one or more outcome parameters. For example, constraining the AIID to a threshold of 10° and the AED to a threshold of 5° sets the relative importance of the implant impingement factor to be greater than the edge-loading impingement factor.

For a total joint arthroplasty procedure, optimization constraints may include: available makes and models of implants; physical constraints, etc. Any constraint or cost function that is clinically and physically relevant to the surgical procedure and the spatial goals of surgical planning may be used.

Surgical Planning Module

In an embodiment, a surgical planning module 120 receives the optimized position of the implant system 118 generated by the KID model module 116, and provides surgical planning functionality to a surgeon via a user interface (e.g. via the user interface module 122). Surgical planning functionality may focus on spatial planning, and include parameters such as: anatomical angles, anatomical distances, implant sizes, implant make, implant model, implant style, implant position and/or angle with respect to anatomical structures, etc. For example, surgical planning may include templating functionality, such as the functionality provided by systems such as the TraumaCAD™ system (Brainlab A G, Munich, DE). In addition to the optimized implant system position 118 provided by the KID model module 116, surgical planning may include the optimized implant system position, kinematic and inverse dynamic analyses provided by the KID model module 116 or dynamic and kinematic analyses, such as those offered in the Corin OPS™ product (Corin Group, Cirencester Gloucestershire, UK). The surgical planning module 120 preferably receives pre-operative or intra-operative medical images, such as x-rays, magnetic resonance imaging (MRI) scans, computed tomography (CT) scans, ultrasound, intra-operative fluoroscopy, or any other modality useful for spatial surgical planning (i.e. planning spatial aspects of the surgical intervention relative to anatomical structures). The surgical planning module 120 may receive medical images using the Digital Imaging and Communications (DICOM) standard, or any other standard. The surgical planning module 120 may be configured to generate a surgical target or range, representing the desired spatial, biomechanical or reconstructive changes due to the surgical procedure.

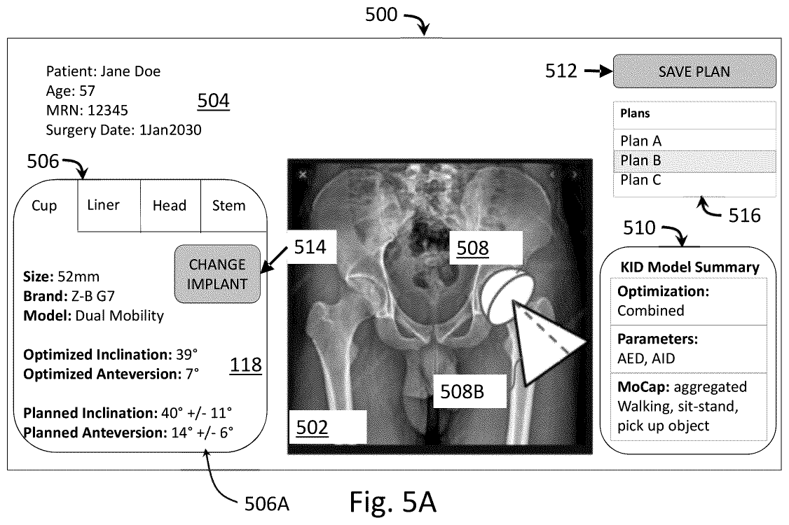

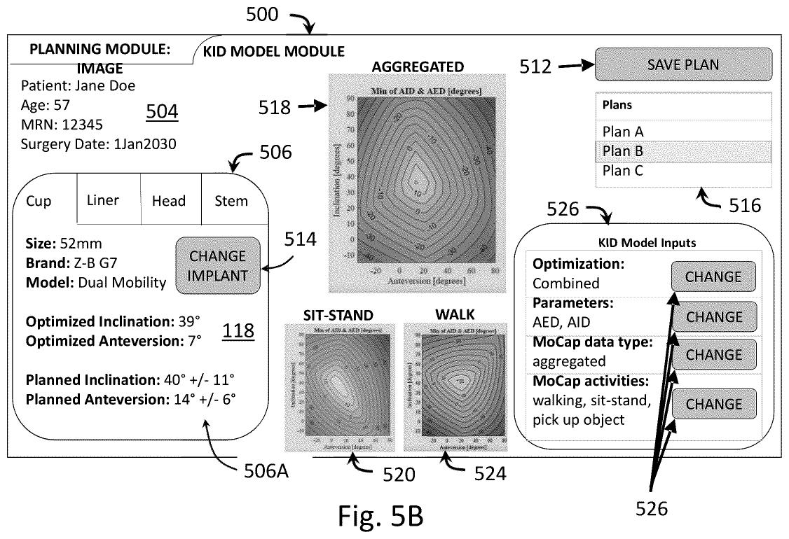

FIG. 5A depicts a user interface (e.g. a GUI) 500 in accordance with an embodiment. The user interface 500 is configured to display a medical image 502 of a patient (by way of example, 502) for whom the surgery is planned. Additional patient details 504, such as name, medical record number (MRN) and surgery date are shown. Planning parameters 506, such as the implant make, model and size are shown. The planning parameters 506 may further include the one or more elements associated with the implant system, such as a cup 308, a liner 310, a femoral stem 304 and a femoral head 306 for use in a total hip arthroplasty. The planning parameters 506 may include the optimized implant system position 118 generated by the KID model module 116, comprising one or more of a cup orientation (anteversion and inclination), a cup translational position (not shown), a femoral stem size (not shown), a femoral version (not shown) and/or a femoral stem offset (not shown). The planning parameters 506 may further include surgical target information 506A, such as target aspects of the implant system, such as cup orientation (shown as inclination and anteversion), femoral stem size (not shown), femoral stem offset (not shown) and femoral version (not shown) for an acetabular prosthesis for a hip replacement surgery. The surgical target information 506A may be presented numerically, for example, as a range of acceptable values for a particular spatial parameter (for example, the acceptable range of acetabular inclination relative to a planning coordinate frame may be between 29° and 51°, or equivalently, 40°+/−11°). Alternatively or in addition, the surgical target information 506A may be presented graphically, as shown in target graphic 508B, in which the shape of the target graphic 508B illustrates angular boundaries of the surgical target relative to the current position of an implant overlay 508 (which includes a dotted line indicating the implant axis, the dotted line lying within the target graphic 508B indicating that the current implant position as indicated by the overlay is within the surgical target zone).

User interface 500 may be further configured to display KID model summary information 510, such as the type of optimization (combined optimization for two or more outcome parameters or optimization of one or more outcome parameters while one or more other outcome parameters are constrained, as discussed above). The KID model summary information 510 may further comprise the outcome parameters included in the KID model 124, and information about the MoCap data 112A, for example, whether the MoCap data 112A is for a single activity or for aggregated activities and which activities are included in the MoCap data 112A.

Alternatively or in addition, the user interface 500 may be configured to display the optimized implant position 118 generated by the KID model module 116 in a graphical format, as shown in FIG. 5B. In an embodiment, the user interface 500 is configured to display the one or more contour plots (shown by example as 518, 520, and 524) generated by the KID model 124 and displaying the values of the one or more outcome parameters as a function of one or more aspects of the implant system position 300. For example, the contour plot 518 may display the minimum of the combined optimization of the angular implant impingement distance and the angular edge-loading distance generated by the KID model module 116 as a function of the cup orientation (anteversion and inclination). The one or more contour plots (shown by example as 518, 520, and/or 524) may be generated based on the application of MoCap data 112A for a single activity (as shown in FIG. 5B for contour plots 520, 524) or for MoCap data 112A that is an aggregate of two or more activities (as shown in FIG. 5B for contour plot 518). In other embodiments, the one or more contour plots (shown by example as 518, 520, and/or 524) may comprise any of the outcome parameters and any of the aspects of the implant system position. Further, the one or more contour plots (shown by example as 518, 520, and 524) may display an aspect of the implant system position for at least one outcome parameter that is optimized and at least one outcome parameter that is constrained, or two or more outcome parameters that may be optimized as a combined optimization. In the embodiment, the user may select how many contour plots (e.g. 518, 520, and/or 524) are displayed. For example, the user may choose to display 1, 2, 3, 4 or more contour plots. The user may further be able to select the size and location of the one or more contour plots (shown by example as 518, 520, and 524).

The user interface 500 may further be configured to display the KID model inputs 526, such as the type of optimization (combined optimization for two or more outcome parameters or optimization of one or more outcome parameters while one or more other outcome parameters are constrained, as discussed above), the outcome parameters included in the KID model 124, the type of MoCap data 112A (e.g. for a single activity or for aggregated activities), and which activities are included in the MoCap data 112A.

In an embodiment, the user interface 500 may be configured according to FIG. 5A and FIG. 5B, where the medical image 502 of a patient and the one or more contour plots (shown by example as 518, 520, and 524) generated by the KID model module 116 are displayed on separate tabs. Alternatively or in addition, the user interface 500 may be configured with the medical image 502 and the one or more contour plots (shown by example as 518, 520, and 524) displayed side-by-side in a single tab (not shown) or top-and-bottom (not shown). In accordance with the embodiment, the user interface 500 may be further configured to allow the user to select how many contour plots are displayed.

In accordance with an embodiment, the surgical planning system 114 facilitates a user to interact with the user interface by providing various controls to conduct planning, such as handles on graphical overlays, such as implant overlays, buttons, data capture fields, menus, drop-down menus, etc. For example, in an embodiment, the user may change or set various options using, for example, buttons. The user may select, for example, the implant make, model or size using the “change implant” button 514 (a UI control).

The surgical planning system 114 further facilitates user interaction with the user interface 500 by providing controls to set and/or change the KID model inputs 526. In accordance with an embodiment, the user interface may provide an input function to input the KID model inputs 526, such as indicated by one or more “change” buttons 526 (a UI control), which prompt the user to select or input various KID model inputs, such as the type of optimization, one or more outcome parameters to be included, and which outcome parameters are optimized or constrained. The user may further select the type of MoCap data 112A (i.e. aggregated versus separate singular activities) and the activities to be included in the MoCap data 112A used by the KID model module 116. Alternatively or in addition, other types of controls can be provided to facilitate a user to interact with the user interface, such as data capture fields, drop-down menus, etc.

In accordance with an embodiment, the surgical planning module 120 transmits the new KID model inputs 526 to the KID model module 116, which in turn generates a new optimized implant system position 118 based on the user's selections and then updates the surgical plan for display (e.g. the surgical target information 506A and the planning parameters 506). In an embodiment, the user may select any of the following: the type of optimization performed, whether the MoCap data 112A comprises one or more single activities or aggregated activities, and which activities are applied to the KID model 124 to generate the optimized implant system position 118. By enabling the user to select the activities, the KID model module 116 enables the user to optimize the implant system position 118 for one or more specific activities. The new optimized implant system position 118 is then provided to the surgical planning system 120.

In accordance with an embodiment, the user may further be able to select a weighting for each activity, which may be based on the desired outcome for the patient. For instance, the patient may prioritize the ability to play golf over the ability to run. The surgeon may assign a greater weighting value to one or more activities compared to one or more other activities based on the desired outcome and/or the preferred activities of the patient. Alternatively, or in addition, the surgeon may assign weighting values to one or more outcome parameters and/or outcome factors to achieve a desired outcome. For example, the surgeon may assign a greater weighting to the implant impingement factor compared to the edge-loading factor and the outcome parameters associated with the edge-loading factor.

In accordance with an embodiment, the surgical planning module 120 feeds back data relating to the surgical plan (e.g. planned implant positions) to the KID model module 116 for recalculation of the optimized implant system position 118. Such a feedback loop may proceed iteratively, in response to a user's changing plan information via the user interface of the planning system 114.

In accordance with an embodiment, the surgical planning module 120 receives the optimized implant system position 118 and the KID model input parameters 526 from the KID model module 116 and may further be configured to provide multiple surgical plans (i.e. surgical target information 506A and/or planning parameters 506). A user interface, in accordance with an embodiment, facilitates a user to select between one or more surgical plans. A surgical plan may be associated with a particular implant make, model and/or size, and/or the KID model inputs 526 (e.g. An optimization type, MoCap data 112A for a specific activity, etc) and/or an optimized implant system position 118 generated by a KID model 124. The user may save a surgical plan, in accordance with an embodiment, for example, using the save plan button 512. The list of plans may be a static table with any number of pre-defined plans with pre-defined names (e.g. Plan A, Plan B, Plan C as shown in the list 516 in FIG. 5A). For example, the static list may comprise 2, 3, 4, 8, 10 plans or any other number. Alternatively, the static table may instead be a drop-down menu or any other type of menu from which the user can select a plan name. In another embodiment, the user may be able to insert in a user-defined plan names in a data capture field when prompted by the user interface 500, for example, when pressing the save plan button 512. The advantage of allowing a user (such as a surgeon) to view surgical plans associated with different implant make, model or size and/or KID model inputs 526 and/or different optimized implant system positions 118 is to provide insight, for example, into the utility of the surgical planning system 114 or into how effective the optimized implant system position 118 will be for the patient while performing different activities post-surgery.

Examples of implant types that may be considered planning parameters 506 include: dual mobility implants or traditional implants. Planning parameters 506 may also include implant materials, specifically the materials of the liner 308 and the cup 310. For example, an implant system 300 including a ceramic liner and a ceramic cup may be more robust to edge-loading because of ceramic's favourable wear properties whereas an implant system 300 including a metal cup and a polyethylene liner may be less robust to edge-loading.

Planning parameters 506 may further include surgical approach information (such as the direct anterior approach, posterior approach and direct lateral approach).

Registration to Image Coordinate System

FIG. 6 is an illustration of data acquisition devices and data, specifically medical image data 608 and MoCap data 112A, that may each have respective coordinate systems and may be coregistered for use in surgical planning, in accordance with an embodiment. With reference to FIG. 6, MoCap data 112A may have an association with a MoCap data coordinate system 604. That is, the spatial data that is collected by the MoCap system 108 may be expressed within a defined 3D coordinate system (or coordinate frame). Prior to surgery, the patient 102 may undergo medical imaging scans (such as x-ray, CT, MRI, ultrasound, etc.) via an imaging device 606, for diagnostic and pre-operative planning purposes, resulting in medical image data 608. The medical image data 608 may have an association with a medical image coordinate system 610. There exists a registration relationship 612 between the MoCap data 112A and the medical image coordinate system 610. The registration relationship 612 may be a rigid body transformation from one coordinate system to the other, and may be expressed mathematically as an affine transformation matrix.

Coordinate systems (e.g. the MoCap data coordinate system 604 and medical image coordinate system 610) may be orthogonal Cartesian systems. Each coordinate system may be defined by the location of its origin and the direction of the basis vectors. Both coordinate systems may represent their respective data (from spatial MoCap data 112A and medical image data 608) in coordinate systems defined by the patient. For example, the patient 102 may have biomechanical or anatomical axes or locations used to define both the MoCap data coordinate system 604 and the medical image coordinate system 610. The biomechanical location may be anatomical landmarks. In another example, the two coordinate systems may be defined differently (i.e. with respect to different anatomical locations or axes), but the different coordinate system definitions may be relatable through rigid body transformations that are known or determinable.

The registration relationship 612 enables the MoCap data 112A and the medical image data 608 to be expressed in a common coordinate frame relative to the patient 102. This is advantageous, since both the medical image data 608 and the MoCap data 112A may be used by the surgical planning module 120. Both coordinate systems may be relatable to a patient coordinate system, such as the standing coronal plane, supine coronal plane, and anterior pelvic plane. The surgical planning module 120 may use the MoCap data 112A and the medical image data 608 relative to a common coordinate system to provide surgical planning functionality (e.g. offering a user interface in which the medical image data 608 and MoCap data 112A, or data derived therefrom, may be visualized and/or manipulated in the same view), or perform surgical planning steps (e.g. an optimized implant system position 118 may be calculated relative to the common coordinate system or frame, during which the surgical planning module 120 may perform spatial optimizations using the medical image data 608 and MoCap data 112A within the same coordinate system).

Converting the MoCap data 112A to a common coordinate system with the medical image data 608 (or vice versa) may be done using mathematical operations using a computer system. For example, in an embodiment, rigid body transformation operations are applied using the registration relationship 612. Such operations may be performed by the surgical planning module 120. The operations may be performed by first determining the medical image coordinate system 610 and the MoCap data coordinate system 604, which may be done in any of the following manners: the respective data may be in an inherent or assumed coordinate system (e.g. the supine coronal coordinate system for a CT scan), the respective data may include further data defining the coordinate system (e.g. fiducial markers present within an x-ray scan, wherein the fiducial markers define the coordinate system, including the case wherein the fiducial markers of the medical image comprise the sensors and/or markers 104 of the MoCap system 108), and the respective data include enough information from which to calculate the respective coordinate system (for example, gait data from inertial and/or IMU sensors may be used to calculate anatomical axes). Where the fiducial markers of the medical image comprise the sensors and/or markers of the MoCap system 108, the fiducial markers may include radiopaque features as coupled to the patient for generating MoCap data 112A. The radiopaque features may be optical markers coupled to the patient (as discussed above) or IMU sensors or other sensors (as discussed above) included in the MoCap system 108, with radiopaque features embedded within. The radiopaque features may have a known or measurable position relative to the MoCap data coordinate system 604 and the medical image data 608 may include an image of the MoCap system 108 radiopaque features as coupled to the patient 102 or embedded within the sensors and/or markers 104 of the MoCap system 108 for generating the MoCap data 112A. Further, the radiopaque features in the medical image coordinate system 610 may be measured using image processing of the medical image data 608.

The registration relationship 612 may be calculated using the respective coordinate frames (of the medical image data 608 and MoCap data 112A) relative to the same physical anatomical features, landmarks or axes. The anatomical features or landmarks may include one or more anterior superior iliac spine (ASIS) points, hip center of rotation, a pubis point, posterior superior iliac spine (PSIS) points, and/or standing plumb line. For the purpose of registering the MoCap data 112A to the medical image coordinate system 610, any number of anatomical landmarks may be used. For instance, the hip center of rotation is commonly used. However, it may not be needed where other anatomical landmarks are used to provide sufficient information to define the registration relationship 612. Calculating the registration relationship may comprise calculating a transformation between the MoCap data coordinate system 604 and the medical image data coordinate system 610 using the locations of the anatomical landmark data in the respective coordinate systems. In another example, the registration relationship 612 may be calculated by calculating a transformation between the MoCap data coordinate system 604 and the medical image coordinate system 610 using the locations of the radiopaque features in the respective coordinate systems.

In accordance with an embodiment, the surgical planning module 120 provides registration and target information to a surgical navigation or robotic system. The registration information may include information useful to a surgical navigation or robotic system to register the patient, such that the navigation or robotic system can be used to achieve the desired target, as defined by the target information (e.g. implant position).

Coregistration to Surgical Navigation System

In an embodiment, an intra-operative navigation system 706 may be used for executing the optimized implant position 118 during a THA, for example, where medical image data 608 is not employed for pre-operative surgical planning. In this embodiment, the MoCap data coordinate system 604 can be registered together with an intra-operative navigation system coordinate system 710.

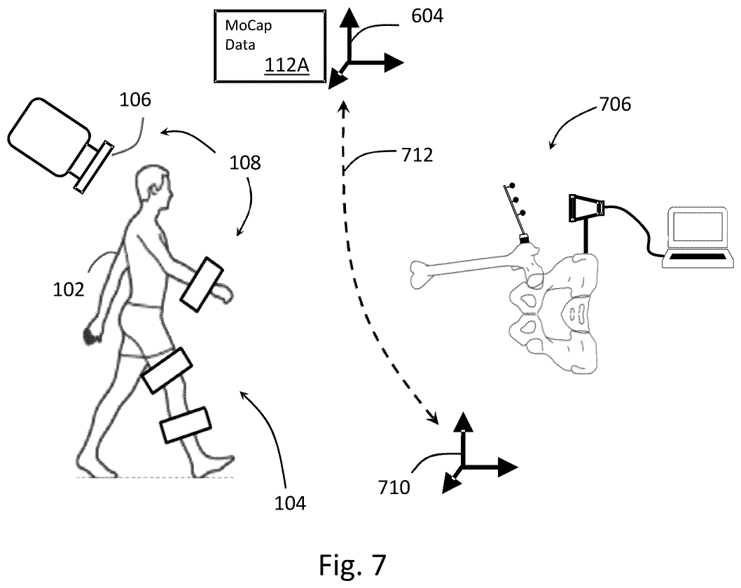

FIG. 7 is an illustration of data acquisition devices and data, specifically surgical navigation data and MoCap data 112A that may each have respective coordinate systems and may be coregistered for use in surgical navigation, in accordance with an embodiment. Intra-operative navigation systems 706 are well known in the art. For example, a method and system for surgical navigation has been disclosed in applicant's U.S. patent U.S. Pat. No. 9,247,998, granted Feb. 2, 2016 and entitled “System and Method of Intra-Operative Leg Position Measurement”, the content of which is incorporated herein by reference in its entirety. The intra-operative navigation system 706 may be used to perform precise measurements intra-operatively to assist the surgeon in executing bone resections and implant placement. As such, the intra-operative navigation system 706 may be associated with anatomical landmark data of the patient, such as the ASIS points, the hip center of rotation, a pubis point, PSIS points and/or the standing plumb line. The navigation system data may further comprise measurements of the patient's anatomy, such as leg length and offset.

Similar to that described for the registration of the MoCap data coordinate system 604 together with the image data coordinate system 610, there exists a registration relationship 712 between the MoCap data coordinate system 604 and the intra-operative navigation system coordinate system 710. The registration relationship may be a rigid body transformation from one coordinate system to the other, and may be expressed mathematically as an affine transformation matrix.

The intra-operative navigation system coordinate system 710 and the MoCap data coordinate system 604 may be orthogonal Cartesian systems, as discussed above, with each coordinate system defined by the location of its origin and the direction of the basis vectors. The MoCap data coordinate system 604 and the intra-operative navigation system coordinate system 710 may represent their data in a coordinate system defined by the patient 102. For example, the patient 102 may have biomechanical or anatomical axes or anatomical landmarks used to define both the MoCap data coordinate system 604 and the navigation system coordinate system 710. As such, the MoCap data 112 may comprise landmark data and the MoCap landmark data may be associated with the same anatomical landmark data as the intra-operative navigation system 706.

The registration relationship 712 enables the MoCap data 112A and therefore the optimized implant position 118 generated by the KID model module 116 by applying the MoCap data 112A and the intra-operative navigation system data to be expressed in a common coordinate frame relative to the patient 102. This is advantageous, as it enables the intra-operative navigation system 706 to be used to execute the optimized implant position 118. Both coordinate systems may be relatable to a patient coordinate system, such as the standing coronal plane, supine coronal plane, and anterior pelvic plane. The intra-operative navigation system 706 may use the MoCap data 112A and the intra-operative navigation system data relative to a common coordinate system for executing the optimized implant position 118.

Converting the MoCap data 112A (and therefore the optimized implant position 118) to a common coordinate system with the intra-operative navigation system 706 (or vice versa) may be done using mathematical operations using a computer system (in particular, by applying rigid body transformation operations using the registration relationship 712). Such operations may be performed by the intra-operative navigation system 706 and/or the KID model module 116. The operations may be performed by first determining the intra-operative navigation system coordinate system 710 and the MoCap data coordinate system 604. The MoCap data coordinate system may be determined using MoCap data 112A which may include further data defining the coordinate system (e.g. fiducial markers present in the MoCap data 112A, such fiducial markers having known relationships to anatomical landmarks and enough information included in the data from which to calculate the respective coordinate system (for example, gait data from inertial sensors may be used to calculate anatomical axes). Alternatively, the MoCap data coordinate system 604 may be defined using a gravity vector and alignment rod to identify the patient's anatomical axes with respect to the fiducial markers. The navigation system coordinate system may be defined by probed landmarks, such as the ASIS, AIIS, and/or the hip center of rotation. The registration relationship 712 may be calculated using the respective coordinate frames relative to the same physical anatomical landmarks or axes. Alternatively, the MoCap data 112A may be collected immediately before surgery using fiducial markers attached to the patient which are then also attached to the patient during surgery.

Computer Device

A computer device comprises a processor and a storage device coupled thereto, which storage device stores instructions for execution by the processor to configure its operations and that of the computer device so as to perform a method. The method may comprise any of the computer implemented methods as described herein. The computing device typically further comprises an input device and an output device. An input device may comprise any of a keyboard, button, pointing device, microphone, camera, sensor (e.g. GPS or other sensors such as described hereinabove), etc. An output device may comprise a display screen, speaker, light, bell, etc. Some devices provide both input and output functions such as a touch screen device. The computing device further typically comprises a communication subsystem and is configured to communicate such as with coupled input or output devices and/or another computing device via wired or wireless means. The processor may comprise a central processing unit (CPU) and/or a graphics processing unit (GPU). The processor may comprise a component of a microcontroller. Storage devices may comprise memory devices including read only memory and random access memory, etc.; hard drives, disc drives, etc. A computer program product comprises a storage device (e.g. a non-transitory device), which stores instructions for execution by a processor of a computing device.

Operations

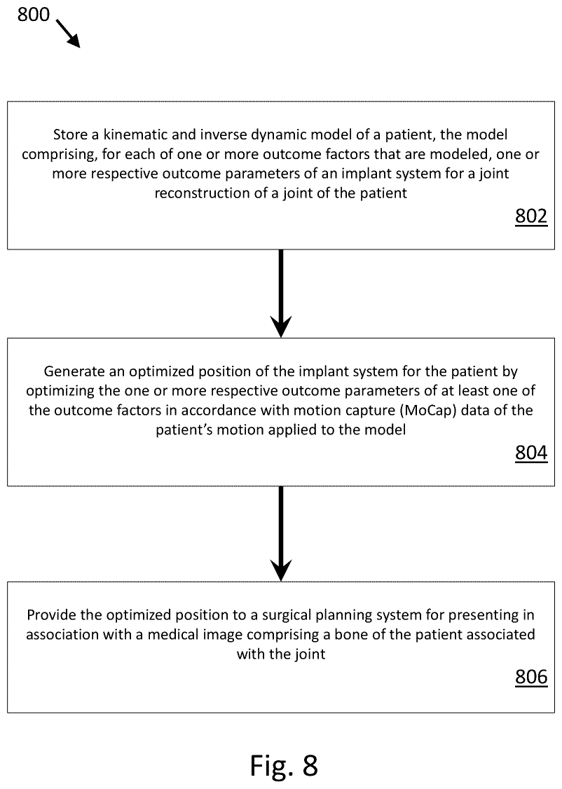

FIG. 8 is a flowchart of operations 800 of a computer implemented method for pre-operative surgical planning a total joint arthroplasty for a patient. At 802 operations stores a KID model comprising, for each of one or more outcome factors that are modeled, one or more respective outcome parameters of an implant system for a joint reconstruction of a joint of the patient. At 804, operations generate an optimized position of the implant system for the patient by optimizing the one or more respective outcome parameters of at least one of the outcome factors. The optimized position of the implant system is generated in accordance with MoCap data of the patient's movement applied to the model. In an embodiment, geometric and inertial parameter data 112B may also be applied to the model to generate the optimized position of the implant system 300. At 806, operations present the optimized implant position 118 in association with a medical image comprising a bone of the patient associated with the joint.

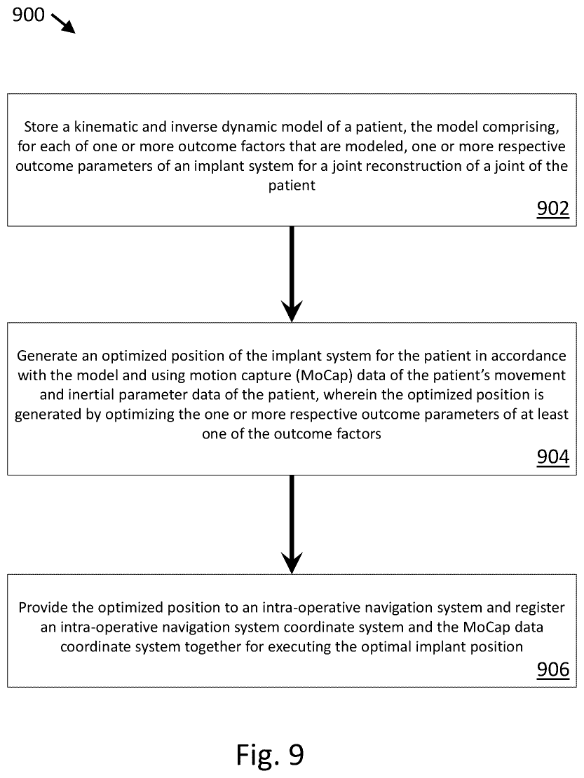

FIG. 9 is a flowchart of operations 900 of a computer implemented method for intra-operative surgical planning a total joint arthroplasty for a patient. At 902 operations stores a KID model comprising, for each of one or more outcome factors that are modeled, one or more respective outcome parameters of an implant system for a joint reconstruction of a joint of the patient. At 904, operations generate an optimized position of the implant system for the patient in accordance with the model and using MoCap data of the patient's movement and geometric and inertial parameter data 112B of the patient, wherein the optimized position is generated by optimizing the one or more respective outcome parameters of at least one of the outcome factors. The MoCap data is in a first coordinate system, and the MoCap data comprises MoCap landmark data associated with anatomical landmarks of the patient spanning the first coordinate system. At 906 operations provide the optimized position, in the first coordinate system, to an intra-operative navigation system, the system configured to: receive intra-operative anatomical landmark data associated with the same anatomical landmarks associated with the MoCap landmark data, and register an intra-operative navigation system coordinate system and the first coordinate system together for executing the optimal implant position.

Practical implementation may include any or all of the features described herein. These and other aspects, features and various combinations may be expressed as methods, apparatus, systems, means for performing functions, program products, and in other ways, combining the features described herein. A number of embodiments have been described. Nevertheless, it will be understood that various modifications can be made without departing from the spirit and scope of the processes and techniques described herein. In addition, other steps can be provided, or steps can be eliminated, from the described process, and other components can be added to, or removed from, the described systems. Accordingly, other embodiments are within the scope of the following claims.

Throughout the description and claims of this specification, the word “comprise” and “contain” and variations of them mean “including but not limited to” and they are not intended to (and do not) exclude other components, integers or steps. Throughout this specification, the singular encompasses the plural unless the context requires otherwise. In particular, where the indefinite article is used, the specification is to be understood as contemplating plurality as well as singularity, unless the context requires otherwise.

Features, integers, characteristics, or groups described in conjunction with a particular aspect, embodiment or example of the invention are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings) or to any novel one, or any novel combination, of the steps of any method or process disclosed.

### REFERENCES (INCORPORATED HEREIN WHERE PERMISSIBLE)

Shuyang Han, Virgenal L Owens, Rikin V Patel, Sabir K Ismaily, Melvyn A Harrington, Stephen J Incavo, Philip C Noble, “The continuum of hip range of motion: From soft-tissue restriction to bony impingement” (22 Jan. 2020), Wiley Online Library, Journal of Orthopaedic Research Vol 38, Issue 8, p 1779-1786.

What is claimed is:

1. A computer-implemented method comprising:
storing a kinematic and inverse dynamic model of a patient, the model comprising, for each of one or more outcome factors that are modeled, one or more respective outcome parameters of an implant system for a joint reconstruction of a joint of the patient;
generating, in accordance with motion capture (MoCap) data for the patient's movement and geometric and inertial parameter data for the patient applied to the model, an optimized position of the implant system for the patient by optimizing the one or more respective outcome parameters of at least one of the outcome factors; and
presenting the optimized position in association with a medical image comprising a bone of the patient associated with the joint.

2. The method of claim 1, wherein the one or more outcome factors comprises any of: an edge loading factor, an implant impingement factor; a bony impingement factor; a bone-on-implant impingement factor; and a soft tissue impingement factor.

3. The method of claim 1, wherein the one or more outcome factors comprises: an edge loading factor and an implant impingement factor.

4. The method of claim 1, wherein generating the optimized position comprises performing a mathematical optimization which minimizes at least some of the respective outcome parameters.

5. The method of claim 1, wherein:
the one or more outcome factors comprises a plurality (N) of outcome factors; and
generating the optimized position comprises constraining the one or more respective parameters respectively associated with N-1 of one or more outcome factors while optimizing the one or more respective outcome parameters associated with one of N outcome factors that is unconstrained.

6. The method of claim 1, wherein generating the optimized position comprises performing, for at least two of the outcome factors, a combined optimization of one or more respective outcome parameters respectively associated with at least two of the outcome factors.

7. The method of claim 1, wherein the model performs an estimation of ground reaction forces and moments without the need for force plate measurements.

8. The method of claim 1, wherein:
the MoCap data is in a first coordinate system, and the medical image is in a second coordinate system; and
the method comprises performing a registration of the first coordinate system and the second coordinate system for presenting the optimized position in association with the medical image.

9. The method of claim 8, wherein:
the MoCap data includes anatomical landmark data for the purpose of performing a registration of the first coordinate system and the second coordinate system;
the medical image includes corresponding anatomical landmark data; and
performing the registration comprises calculating a transformation between the first coordinate system and the second coordinate system using locations of the anatomical landmark data in the first coordinate system and respective locations of the corresponding anatomical landmark data in the second coordinate system.

10. The method claim 1, wherein the MoCap data is generated by a MoCap system, wherein the MoCap system uses any of the following technologies: optical marker-based motion capture; marker-less motion capture based on video feed; inertial sensors; and inertial measurement units.

11. The method of claim 10, wherein the MoCap system comprises one or more optical and/or inertial devices having radiopaque features associated with the first coordinate system, and wherein the medical image includes an image of the radiopaque features of the one or more optical and/or inertial devices as coupled to the patient for generating the MoCap data.

12. The method of claim 11, wherein the radiopaque features are one of: optical markers coupled to the patient; an inertial device with radiopaque features embedded within, wherein the radiopaque features comprise at least three retroreflective markers with a known position relative to the MoCap data coordinate system.

13. The method of claim 12, wherein performing the registration comprises calculating a transformation between the first coordinate system and the second coordinate system using locations of the radiopaque features measurable within the MoCap data in the first coordinate system and respective locations of the radiopaque features measurable within the medical image in the second coordinate system.

14. The method of claim 13, wherein the respective locations of the radiopaque features in the second coordinate system are measured using image processing of the medical image.

15. The method claim 1, wherein geometric and inertial parameter data comprise one or more of: body segment lengths, body segment masses, body segment centers of mass, and an inertia matrix.

16. The method of claim 1, wherein the joint is a hip and the implant system is an artificial hip joint comprising any of: a cup; a liner; a stem; and a femoral head.

17. The method of claim 16, wherein the optimized position of the implant system is associated with any of:
a cup orientation;
a cup translational position;
a femoral version;
a femoral head size;
a cup size;
a stem size;
a stem offset; and
a femoral neck-shaft angle.

18. The method of claim 1, wherein the patient image comprises one of an x-ray, a magnetic resonance imaging (MRI) scan, a computed tomography (CT) scan, and an ultrasound scan.

19. The method claim 1 comprising providing the optimized implant position for use by one or both of a surgical planning system, or an intra-operative navigation system.

20. A computer-implemented method comprising:
storing a kinematic and inverse dynamic model of a patient, the model comprising, for each of one or more outcome factors that are modeled, one or more respective outcome parameters of an implant system for a joint reconstruction of a joint of the patient;
generating an optimized position of the implant system for the patient in accordance with the model and using motion capture (MoCap) data for the patient's movement and geometric and inertial parameter data for the patient, wherein: the optimized position is generated by optimizing the one or more respective outcome parameters of at least one of the outcome factors, the MoCap data is in a first coordinate system, and the MoCap data comprises MoCap landmark data associated with anatomical landmarks of the patient spanning the first coordinate system; and
providing the optimized position, in the first coordinate system, to an intra-operative navigation system, the system configured for use when registered together with the first coordinate system for executing the optimal implant position.

21. A method comprising:
storing a kinematic and inverse dynamic model of a patient, the model modeling a plurality of outcome factors and, for each outcome factor that is modeled, the model comprising one or more respective outcome parameters of an implant system for a joint reconstruction of a joint of the patient;
applying motion capture (MoCap) data of the patient's movement and geometric and inertial parameter data of the patient to the model to generate an optimized position of the implant system for the patient, the optimized position generated by optimizing the one or more respective outcome parameters of at least two of the outcome factors; and
providing the optimized position for presenting in association with a medical image comprising a bone of the patient associated with the joint.
